(19) United States
(12) Patent Application Publication
(10) Pub. No.: US 2015/0004261 A1
(43) Pub. Date: Jan. 1, 2015

(54) Rhizoma Arisaematis Extracts and Uses Thereof for Wound-Healing Effects

(71) Applicants: Nancy Yuk-Yu Ip, Hong Kong (CN); Yu Pong Ng, Hong Kong (CN); Fanny Chui Fun Ip, Hong Kong (CN); Shengjun Guo, Hong Kong (CN)

(72) Inventors: Nancy Yuk-Yu Ip, Hong Kong (CN); Yu Pong Ng, Hong Kong (CN); Fanny Chui Fun Ip, Hong Kong (CN); Shengjun Guo, Hong Kong (CN)

(73) Assignee: THE HONG KONG UNIVERSITY OF SCIENCE AND TECHNOLOGY, Hong Kong (CN)

(21) Appl. No.: 14/352,954

(22) PCT Filed: Oct. 18, 2012
(86) PCT No.: PCT/CN2012/001399

§ 371 Date: Apr. 18, 2014

Related U.S. Application Data
(60) Provisional application No. 61/627,772, filed on Oct. 18, 2011.

(51) Int. Cl. A61K 36/8884; A61K 9/00; A61K 31/7028; A61K 31/7032; A61K 31/7076

(52) U.S. Cl. CPC A61K 36/8884; A61K 31/7032; A61K 31/7076; A61K 31/7028; A61K 9/0014
USPC 424/725; 514/25; 536/29.1

(57) ABSTRACT

Provided are water and alcohol extracts of Rhizoma arisaematis, compounds isolated from Rhizoma arisaematis (such as 3-O-(9,12-octadecadienoyl)-glyceryl-β-D-galactopyranoside, N-(β-D-ribofuranos-1-yl)-phenylalanine, and adenosine), pharmaceutical compositions comprising the aforementioned ingredients, as well as uses thereof for promoting wound healing. In one embodiment, the pharmaceutical composition is a topical formulation, such as a cream, ointment, foam, lotion, plaster, gel, and emulsion.

### CROSS-REFERENCE TO RELATED APPLICATION

This application claims the benefit of U.S. provisional application Ser. No. 61/627,772, filed Oct. 18, 2011, which is herein incorporated by reference in its entirety.

### BACKGROUND OF THE INVENTION

Rhizoma arisaematis (RA) is a perennial herb in the Araceae family and native to North America and Asia. In traditional Chinese medicine (TCM), RA is considered to have bitter, pungent, and warm properties, and has been used to treat various conditions including cough,muscle spasms, and epilepsy.

Wound-healing is a complex process involving inflammation, cell proliferation, tissue granulation, re-epithelialization, and tissue reorganization; and it is mediated by keratinocytes, fibroblasts, endothelial cells, and immune cells. Wound repair begins with an increase in blood flow to the site of injury and an increase in vascular permeability. Blood constituents leak into the wound and form a hemostatic plug consisting of platelets and extra-cellular matrix (ECM). Neutrophils also mount non-specific but highly effective and destructive phagocytic response. Antigen-specific immune responses are also mediated by the newly arrived monocytes and lymphocytes, which release additional pro-inflammatory cytokines.

Endothelial cells are activated during the initial phase of the inflammatory response during wound healing. The endothelial cells express, among other things, adhesion molecules that attach circulating leukocytes to inflammatory cells. Cellular attachment of immune cells to blood vessels of the endothelial cell lining surrounding the inflammatory site prevents the immune cells from being swept past the site of tissue damage; this is a crucial step for subsequent migration of immune cells into the surrounding inflammatory tissues.

Endothelial cells also degrade existing vascular base membrane and initiate the migration and proliferation of fibroblasts in the wounded area. Proliferating fibroblasts migrate along the fibrin clot into the wound bed and initiate the formation of extracellular matrix known as ground substances. Once the ground substance is laid down, fibroblasts produce collagen, proteoglycan, fibronectin and glucosaminoglycan, resulting in the formation of a new extracellular matrix around the wound bed; the newly formed extracellular matrix is useful for the migration of other cells.

Meanwhile, granulation tissue forms during the wound healing process to cover the wound bed. In addition, keratinocytes migrate and proliferate from the edge of the wound, followed by fibroblast proliferation in the proximal end of the wound. Collagen fibrils are reorganized on the surface, and actin contained in myofibroblasts pulls the wound edges closer thereby reducing the size of the wound.

During wound healing, keratinocytes, fibroblasts, and endothelial cells play roles interdependent on each other. Keratinocyte migration and proliferation are essential in re-epithelialization, which is facilitated by the formation of extra-cellular matrix. Synthesis of new extracellular matrix is also mediated by the proliferation of fibroblasts, which promote the proliferation of keratinocytes. Endothelial cells are involved in angiogenesis, which is critical for supplying cytokines, oxygen, and nutrients to proliferating keratinocytes and fibroblasts. Endothelial cells also provide immune protection throughout the wound-healing process.

Therapeutic agents that promote the migration and/or proliferation of endothelial cells, keratinocytes and/or fibroblasts would be useful for promoting wound-healing, including the healing of acute and chronic wounds (e.g., diabetic wounds).

Currently, REGRANEX® (Becaplermin), a recombinant human platelet-derived growth factor-BB (rhPDGF-BB), is the only growth factor approved by the FDA for the treatment of chronic wounds, in particular, diabetic foot ulcers. There is a need for developing additional therapeutics for wound-healing.

### BRIEF SUMMARY OF THE INVENTION

In various embodiments, the present invention provides water and alcohol extracts of Rhizoma arisaematis (RA), the water-soluble fraction of the Rhizoma arisaematis alcohol extract, compounds isolated from Rhizoma arisaematis (such as 3-O-(9,12-octadecadienoyl)-glyceryl-β-D-galactopyranoside, N-(β-D-ribofuranos-1-yl)-phenylalanine, and adenosine), pharmaceutical compositions comprising the aforementioned ingredients, as well as uses thereof for promoting wound healing. In certain embodiments, the pharmaceutical composition is a topical formulation, such as a cream, ointment, foam, lotion, plaster, gel, or emulsion.

In one specific embodiment, the present invention provides a method for promoting healing of a wound in a subject, wherein the method comprises topically administering to a wound area of the subject, a therapeutically effective amount of a pharmaceutical composition comprising a water-soluble extract of Rhizoma arisaematis, and/or one or more compounds isolated from Rhizoma arisaematisor salts thereof, wherein the method promotes closure and/or healing of the wound.

In another embodiment, the present invention provides a novel compound N-(β-D-ribofuranos-1-yl)-phenylalanine, which can be isolated from Rhizoma arisaematis.

In certain embodiments, the Rhizoma arisaematis extracts of the invention and/or compounds isolated from RA can be used to promote the healing of wounds including skin wounds, excisions, lacerations, burns, abrasions, puncture or penetrating wounds, surgical wounds, contusions, hematomas, crushing injuries, and ulcers, such as diabetic foot ulcers.

### BRIEF DESCRIPTION OF THE DRAWINGS

FIG. 1A shows that the water fraction of Rhizoma arisaematis (RA) closes in vitro scratch wounds in adult human keratinocytes. FIG. 1B shows a quantitative analysis of the width of wound beds after treatment.

FIG. 2 shows that the crude extract (T) and water fraction (WA) of RA induce collagen type I production in human adult fibroblasts.

FIG. 3 shows that the crude extract (T) and water fraction (WA) of RA do not cause cell death in human neonatal keratinocytes.

FIG. 4 shows a proliferative effect of WA fraction of Rhizoma arisaematis as revealed by MTT (3[4,5-dimethylthiazol-2-yl]-2,5-diphenyltetrazolium bromide) metabolic activity.

FIG. 5 shows water sub-fractions of RA, RA-WA1 and RA-WA2, close in vitro scratch wounds in adult human keratinocytes.

FIG. 6A shows effects of WA fraction and RA-WA1 on wound closure on day 2.

FIG. 6B shows effects of RA-WA1 at 4 and 40 mg/kg on wound closure for a period of 8 days.

FIG. 6C shows the wound-healing effects of RA-WA1 at 40 mg/kg or water. The mouse was topically treated by RA-WA1 and water, respectively, on one wound; while the other wound was left untreated. The picture was taken on day 8.

FIG. 7 shows HPLC profiling of the RA-WA fraction and the isolated compounds. UV absorbance was detected at 254 nm.

FIG. 8A shows the flow chart of isolation of compound G192-C07. FIG. 8B shows that 3-O-(9,12-octadecadienoyl)-glyceryl-β-D-galactopyranoside (compound G192-C07), isolated from Rhizoma arisaematis, closes in vitro scratch wound in adult human keratinocytes.

### DETAILED DISCLOSURE OF THE INVENTION

In various embodiments, the present invention provides water and alcohol extracts of Rhizoma arisaematis (RA), the water-soluble fraction of the Rhizoma arisaematis alcohol extract, compounds isolated from Rhizoma arisaematis (such as 3-O-(9,12-octadecadienoyl)-glyceryl-β-D-galactopyranoside, N-(β-D-ribofuranos-1-yl)-phenylalanine, and adenosine), pharmaceutical compositions comprising the aforementioned ingredients, as well as uses thereof for promoting wound healing. In certain embodiments, the pharmaceutical composition is a topical formulation, such as a cream, ointment, foam, lotion, plaster, gel, or emulsion.

Species of Rhizoma arisaematis include Arisaema erubescens, Arisaema heterophyllum, and Arisaema amurense. In one embodiment, the present invention provides Arisaema amurense extracts and biologically-active chemical constituents (such as 3-O-(9,12-octadecadienoyl)-glyceryl-β-D-galactopyranoside, N-(β-D-ribofuranos-1-yl)-phenylalanine, and adenosine) isolated from Arisaema amurense, and uses thereof for promoting wound healing.

In certain embodiments, the present invention provides extracts and biologically-active chemical constituents (such as 3-O-(9,12-octadecadienoyl)-glyceryl-β-D-galactopyranoside, N-(β-D-ribofuranos-1-yl)-phenylalanine, and adenosine) isolated from Arisaema erubescens and/or Arisaema heterophyllum, and uses thereof for promoting wound healing.

In one embodiment, the Rhizoma arisaematis extracts and/or compounds isolated from RA can be used to promote the proliferation and/or migration of keratinocytes, and/or the production of collagen by fibroblasts.

In certain embodiments, the Rhizoma arisaematis extracts of the invention and/or compounds isolated from RA can be used to promote the healing of wounds including skin wounds, excisions, lacerations, burns, abrasions, puncture or penetrating wounds, surgical wounds, contusions, hematomas, crushing injuries, and ulcers, such as diabetic foot ulcers.

### Compounds

In one embodiment, the present invention provides N-(β-D-ribofuranos-1-yl)-phenylalanine, or a salt thereof. The N-(β-D-ribofuranos-1-yl)-phenylalanine compound has the following structure:

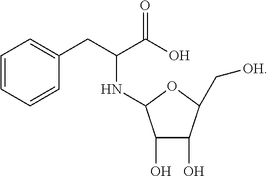 [SMILES: O=C(O)C(Cc1ccccc1)NC1OC(CO)C(O)C1O]

In another embodiment, the present invention pertains to 3-O-(9,12-octadecadienoyl)-glyceryl-β-D-galactopyranoside and adenosine, and salts thereof. In one specific embodiment, the present invention pertains to 3-O-(9Z,12Z-octadecadienoyl)-glyceryl-β-D-galactopyranoside. In one specific embodiment, the present invention pertains to 3-O-(9,12-octadecadienoyl)-glyceryl-β-D-galactopyranoside, N-(β-D-ribofuranos-1-yl)-phenylalanine, and adenosine isolated from Rhizoma arisaematis. In one embodiment, the present invention pertains to 3-O-(9,12-octadecadienoyl)-glyceryl-β-D-galactopyranoside, N-(β-D-ribofuranos-1-yl)-phenylalanine, and adenosine isolated from Arisaema amurense.

### Rhizoma Arisaematis Extracts

In another aspect, the present invention provides Rhizoma arisaematis extracts, as well as methods for preparing Rhizoma arisaematis extracts and for isolating biologically-active chemical constituents from Rhizoma arisaematis. Also provided are Rhizoma arisaematis extracts prepared in accordance with the subject invention. In one embodiment, the present invention provides Arisaema amurense extracts and biologically-active chemical constituents isolated from Arisaema amurense. In one embodiment, the present invention provides a polar solvent soluble extract of Rhizoma arisaematis. Examples of polar solvents useful for preparing the Rhizoma arisaematis extract of the invention include water, isopropanol, n-propanol, ethanol, methanol, n-butanol, isobutanol, and various mixtures thereof.

In one specific embodiment, the present invention provides a polar solvent soluble extract of Rhizoma arisaematis, wherein the polar solvent is selected from water, a C1-C4 alcohol (e.g., methanol, ethanol, n-propanol, isopropanol, n-butanol, isobutanol) or a mixture of C1-C4 alcohols, or a water-C1-C4 alcohol mixture. In one further specific embodiment, the present invention provides a water-soluble extract of Rhizoma arisaematis. In one embodiment, the present invention provides a water-soluble fraction of the polar solvent soluble extract of Rhizoma arisaematis, wherein the polar solvent is selected from water, a C1-C4 alcohol (e.g., methanol, ethanol, n-propanol, isopropanol, n-butanol, isobutanol) or a mixture of C1-C4 alcohols, or a water-C1-C4 alcohol mixture. In a preferred embodiment, the Rhizoma arisaematis extracts of the invention are extracted from the roots of Rhizoma arisaematis.

In one embodiment, the present invention provides a method for preparing Rhizoma arisaematis extract and/or for isolating biologically-active chemical constituents from Rhizoma arisaematis, wherein the method comprises, consists essentially of, or consists of the steps of:
a) providing a sufficient quantity of raw material of Rhizoma arisaematis; and
b) extracting the raw material of Rhizoma arisaematis with a solvent that comprises a C1-C4 alcohol (e.g., methanol, ethanol, propanol, isopropanol, butanol) to yield an alcohol soluble extract of Rhizoma arisaematis extract.

Preferably, the raw material of Rhizoma arisaematis is dried and smashed to small pieces. In a further embodiment, the raw material of Rhizoma arisaematis is immersed in a solvent under reflux.

In one embodiment, the solvent for preparing the alcohol extract comprises, or is, an alcohol-water mixture. The alcohol-water (e.g., ethanol-water, methanol-water) mixture can comprise at least (v/v) 5%, 10%, 15%, 20%, 25%, 30%, 35%, 40%, 50%, 55%, 60%, 65%, 70%, 75%, 80%, 85%, 90%, or 95% alcohol (e.g., ethanol, methanol, n-propanol, isopropanol, n-butanol, isobutanol).

In a further embodiment, the present invention provides a water-soluble fraction of the alcohol (e.g., ethanol) extract of Rhizoma arisaematis. In another further embodiment, the present invention provides a butanol-soluble fraction of the ethanol extract of Rhizoma arisaematis.

In one embodiment, the present invention provides a water-soluble extract of Rhizoma arisaematis, wherein the water-soluble extract comprises one or more of the following compounds: uracil, uridine, adenine, adenosine, guanine, isoguanosine, N-(β-D-ribofuranos-1-yl)-phenylalanine, phenylalanine, leucylphenylalanine, 2,6-deoxyfructosazine, tyrosine, 3-O-(9Z,12Z-octadecadienoyl)-glyceryl-β-D-galactopyranoside, β-D-fructofuranosyl-(2→5)-fructopyranose, and β-D-fructofuranose/β-D fructopyranose. The Rhizoma arisaematis extract can be collected by, for example, filtration to remove the residues. In one embodiment, the Rhizoma arisaematis extract may be further evaporated to produce solid or semi-solid compositions. In another embodiment, the Rhizoma arisaematis extract may be concentrated and/or purified.

In a further embodiment, the subject method comprises creating a chemical profile for the Rhizoma arisaematis extract, using techniques such as NMR analysis and chromatography, for example, high-performance liquid chromatography (HPLC) and hydrophilic interaction liquid chromatography (HILIC).

In another embodiment, the present invention provides a method of isolating 3-O-(9,12-octadecadienoyl)-glyceryl-β-D-galactopyranoside from Rhizoma arisaematis, wherein the method comprises:
providing a butanol-soluble extract of the root of Rhizoma arisaematis; and
isolating the 3-O-(9,12-octadecadienoyl)-glyceryl-β-D-galactopyranoside from the butanol-soluble extract of the root of Rhizoma arisaematis. In one embodiment, 3-O-(9,12-octadecadienoyl)-glyceryl-β-D-galactopyranoside is isolated from Rhizoma arisaematis in accordance with the present invention.

In another embodiment, the present invention provides a method of isolating N-(β-D-ribofuranos-1-yl)-phenylalanine from Rhizoma arisaematis, wherein the method comprises:
providing a water-soluble extract of the root of Rhizoma arisaematis; and
isolating the N-(β-D-ribofuranos-1-yl)-phenylalanine from the water-soluble extract of the root of Rhizoma arisaematis.

The term “consisting essentially of,” as used herein, limits the scope of the invention to the specified steps and those that do not materially affect the basic and novel characteristic(s) of the subject invention, i.e., a method for obtaining Rhizoma arisaematis extract and/or for isolating biologically-active chemical constituents from Rhizoma arisaematis. By using “consisting essentially of,” the method for preparing Rhizoma arisaematis extract does not contain any unspecified steps of extracting or contacting Rhizoma arisaematis with unspecified solvent(s). However, by using the term “consisting essentially of,” the process may comprise steps that do not materially affect the extraction of biologically-active chemical constituents from Rhizoma arisaematis including collecting or recovering the Rhizoma arisaematis extract; concentrating the Rhizoma arisaematis extract; combining multiple Rhizoma arisaematis extracts into a single composition; lyophilizing or drying the Rhizoma arisaematis extract into a solid or semi-solid composition; formulating the Rhizoma arisaematis extract into a pharmaceutical composition such as solutions, suspensions, tablets, capsules, granules, powders, decoctions, and tinctures; mixing the Rhizoma arisaematis extract with pharmaceutically-acceptable carriers, excipients, flavoring agents, buffering agents, and/or emulsifying agents; and packaging the Rhizoma arisaematis extract.

### Promotion of Wound Healing

Another aspect of the present invention provides therapeutic uses of a C1-C4 alcohol soluble extract of Rhizoma arisaematis, a water-soluble extract of Rhizoma arisaematis, isolated compounds including 3-O-(9,12-octadecadienoyl)-glyceryl-β-D-galactopyranoside, N-(β-D-ribofuranos-1-yl)-phenylalanine, and adenosine, and salts thereof, as well as therapeutic compositions comprising one or more of the aforementioned ingredients, for promoting wound healing. In one specific embodiment, the present invention provides use of isolated 3-O-(9,12-octadecadienoyl)-glyceryl-β-D-galactopyranoside for promotion of wound healing.

In one embodiment, the present invention provides a method for promoting healing of a wound in a subject, wherein the method comprises administering to a subject having a wound, a therapeutically effective amount of a pharmaceutical composition comprising a C1 -C4 alcohol soluble extract of Rhizoma arisaematis, a water-soluble extract of Rhizoma arisaematis, and/or one or more isolated compounds that are 3-O-(9,12-octadecadienoyl)-glyceryl-β-D-galactopyranoside, N-(β-D-ribofuranos-1-yl)-phenylalanine, or adenosine, or salts thereof.

In certain embodiments of the pharmaceutical composition, the weight percentage of the Rhizoma arisaematis extract is at least 50%, or any weight percentage higher than 50%, including but not limited to, higher than 60%, 65%, 70%, 75%, 80%, 85%, 90%, 95%, 96%, 97%, 98%, or 99% (w/w).

In one embodiment, the composition is administered via the topical route. In one embodiment, the composition is topically administered to a wound area, such as the skin wound or the skin area above the wounded tissue (e.g., wounded subcutaneous tissue).

The term “wound,” as used herein, includes acute and chronic wounds, as well as open and closed wounds. Examples of wounds that can be treated in accordance with the present invention include skin wounds, excisions, lacerations, burns, abrasions, puncture or penetrating wounds, surgical wounds, crushing injuries, and ulcers (such as diabetic ulcers, burn ulcers, traumatic ulcers, or other chronic ulcers).

In one specific embodiment, the present invention provides a method for promoting healing of a wound in a subject, wherein the method comprises topically administering to a wound area of the subject, a therapeutically effective amount of a pharmaceutical composition comprising a water-soluble extract of Rhizoma arisaematis, and/or one or more isolated compounds that are 3-O-(9,12-octadecadienoyl)-glyceryl-β-D-galactopyranoside, N-(β-D-ribofuranos-1-yl)-phenylalanine, or adenosine, or salts thereof, wherein the method promotes closure and/or healing of the wound.

In one embodiment, the present invention provides a method for promoting healing of a wound in a subject, wherein the method comprises topically administering to a wound area of the subject, a therapeutically effective amount of a pharmaceutical composition comprising a water-soluble extract of Rhizoma arisaematis, wherein the water-soluble extract comprises one or more of the following compounds: uracil, uridine, adenine, adenosine, guanine, isoguanosine, N-(β-D-ribofuranos-1-yl)-phenylalanine, phenylalanine, leucylphenylalanine, 2,6-deoxyfructosazine, tyrosine, 3 -O-(9Z,12Z-octadecadienoyl)-glyceryl-β-D-galactopyranoside, and β-D-fructofuranose /β-D fructopyranose.

In one specific embodiment, the present invention provides a method for promoting healing of a wound in a subject, wherein the method comprises topically administering to a wound area of the subject, a therapeutically effective amount of a pharmaceutical composition comprising an isolated compound that is 3-O-(9,12-octadecadienoyl)-glyceryl-β-D-galactopyranoside and/or an isolated compound that is adenosine, or salts thereof, wherein the method promotes closure and/or healing of the wound. The term “subject,” as used herein, describes an organism, including mammals such as primates, to which treatment with the compositions according to the present invention can be provided. Mammalian species that can benefit from the disclosed methods of treatment include, but are not limited to, apes, chimpanzees, orangutans, humans, monkeys; domesticated animals such as dogs, cats, horses, cattle, pigs, sheep, goats, chickens; and other animals such as mice, rats, guinea pigs, and hamsters.

The term “effective amount,” as used herein, refers to an amount that is capable of promoting wound healing or otherwise capable of producing an intended therapeutic effect.

In another embodiment, the present invention provides a method of promoting the migration and/or proliferation of keratinocytes, wherein the method comprises: administering to the keratinocytes, an effective amount of a composition comprising a C1-C4 alcohol soluble extract of Rhizoma arisaematis, a water-soluble extract of Rhizoma arisaematis, and/or one or more isolated compounds selected from 3-O-(9,12-octadecadienoyl)-glyceryl-β-D-galactopyranoside, N-(β-D-ribofuranos-1-yl)-phenylalanine, adenosine, and any salts thereof.

In one specific embodiment, the present invention promotes the migration and/or proliferation of keratinocytes in a subject.

In another embodiment, the present invention provides a method of promoting collagen production by fibroblasts, wherein the method comprises: administering to the fibroblasts, an effective amount of a composition comprising a C1-C4 alcohol soluble extract of Rhizoma arisaematis, a water-soluble extract of Rhizoma arisaematis, and/or an isolated compound that is 3-O-(9,12-octadecadienoyl)-glyceryl-β-D-galactopyranoside or a salt thereof.

In one specific embodiment, the present invention promotes the production of collagen by fibroblasts in a subject.

In one embodiment, the C1-C4 alcohol soluble extract of Rhizoma arisaematis is soluble in C1-C4 alcohol, or a water-C1-C4-alcohol mixture. The water-C1-C4-alcohol mixture (e.g., ethanol-water, methanol-water) mixture can comprise at least (v/v) 5%, 10%, 15%, 20%, 25%, 30%, 35%, 40%, 50%, 55%, 60%, 65%, 70%, 75%, 80%, 85%, 90%, or 95% C1-C4-alcohol (e.g., ethanol, methanol, n-propanol, isopropanol, n-butanol, isobutanol).

### Therapeutic Compositions, Formulations, and Routes of Administration

In another aspect, the present invention provides a pharmaceutical composition for promoting wound healing. In one embodiment, the pharmaceutical composition is formulated for topical application. In certain embodiments, the pharmaceutical composition of the present invention is formulated as a wound dressing, cream, ointment, foam, lotion, plaster, gel, emulsion, hydrogel, or skin patch.

In one embodiment, the present invention provides for pharmaceutical compositions comprising a therapeutically effective amount of the Rhizoma arisaematis extract of the present invention, and, optionally, a pharmaceutically acceptable carrier. In one specific embodiment, the weight percent of the Rhizoma arisaematis extract in the wound-healing composition is higher than 50% by weight, or any percentages (w/w) higher than 50%, including higher than 60%, 65%, 70%, 75%, 80%, 85%, 90%, 95%, 96%, 97%, 98%, or 99% (w/w). The present invention also provides therapeutic or pharmaceutical compositions comprising compounds isolated from Rhizoma arisaematis (e.g., 3 -O-(9,12-octadecadienoyl)-glyceryl-β-D-galactopyranoside, N-(β-D-ribofuranos-1-yl)-phenylalanine, and adenosine,), or salts thereof.

The term “carrier” refers to a diluent, adjuvant, excipient, or vehicle with which the compound is administered. Such pharmaceutical carriers can be sterile liquids, such as water and oils, including those of petroleum oil such as mineral oil; vegetable oil such as peanut oil, soybean oil, and sesame oil; animal oil; or oil of synthetic origin. Saline solutions and aqueous dextrose and glycerol solutions can also be employed as liquid carriers, particularly for injectable solutions.

The therapeutic or pharmaceutical compositions of the invention can be formulated as neutral or salt forms. Pharmaceutically acceptable salts include, but are not limited to, salts formed with hydrochloric, phosphoric, acetic, oxalic, tartaric acids, sodium, potassium, ammonium, calcium, ferric hydroxides, etc.

The invention also provides a pharmaceutical pack or kit comprising one or more containers filled with one or more of the ingredients, e.g., extracts, compounds, carriers, of the pharmaceutical compositions of the invention.

The compositions of the present invention can also be formulated consistent with traditional Chinese medicine practices. The composition and dosage of the formulations that are effective in the treatment of a particular disease, condition, or disorder will depend on the nature of the disease, condition, or disorder by standard clinical techniques.

The traditional Chinese medicine in prescription amounts can be readily made into any form of drug suitable for administering to humans or animals. Suitable forms include, for example, tinctures, decoctions, and dry extracts. All of the above-mentioned methods are known to people skilled in the art, described in books, and commonly used by practitioners of herbal medicine.

An extract is a concentrated preparation of the essential constituents of a medicinal raw material. Typically, the essential constituents are extracted from the raw medicinal materials (e.g. herbs) by suspending the raw medicinal materials in an appropriate choice of solvent. The extracting process may be further facilitated by means of maceration, percolation, repercolation, counter-current extraction, turbo-extraction, or by carbon-dioxide hypercritical (temperature/pressure) extraction. After filtration to rid of herb debris, the extracting solution may be further evaporated and thus concentrated to yield a soft extract and/or eventually a dried extract, extractum siccum, by means of spray drying, vacuum oven drying, fluid-bed drying, or freeze-drying. The soft extract or dried extract may be further dissolved in a suitable liquid to a desired concentration for administering or processed into a form such as creams, ointments, pills, capsules, etc.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic composition, if desired, can also contain minor amounts of wetting or emulsifying agents, or pH buffering agents. These compositions can take the form of solutions, suspensions, emulsion tablets, capsules, granules, powders, sustained-release formulations and the like. The composition can be formulated with traditional binders and carriers such as triglycerides. Examples of suitable pharmaceutical carriers are described in “Remington's Pharmaceutical Sciences” by E. W. Martin. Such compositions contain a therapeutically effective amount of the therapeutic composition, together with a suitable amount of carrier so as to provide the form for proper administration to the patient. The formulation should suit the mode of administration.

The extracts, compounds, and compositions of the present invention can be administered to the subject being treated by standard routes, including topical, oral, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, and electroporation, as well as co-administration as a component of any medical device or object to be inserted (temporarily or permanently) into a subject. In preferred embodiments, the compositions of the present invention are administered to a subject by topical administration.

The amount of active ingredient that may be combined with the carrier materials to produce a single dosage form will vary, depending on the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

### Materials and Methods

### Plant Materials

The roots of Arisaema amurense (a subspecies of Rhizoma arisaematis), harvested in Heilongjiang province, China, were purchased from Lee Hoong Kee Ltd. (Hong Kong).

### Chemical Fractionation

Chemical fractionation was conducted to obtain extracts and fractions from Rhizoma arisaematis. The raw herb Rhizoma arisaematis (17 kg) was dried in an oven at 60° C. for 2 hours, smashed to small pieces, and immersed in 70% EtOH/H₂O for 30 mins (material to solvent ratio=1:5) before solvent extraction.

The extraction was performed with reflux apparatus for 2 hours and repeated for 3 times to provide a crude solvent extract (T, 950 g). The fractions were then sequentially generated by subjecting the crude extract RA-T to solvents of different polarity. RA-T was first dissolved in water (1:5 by volume), partitioned with chloroform (1:1 by volume) three times, then pooled together and concentrated, thereby yielding the chloroform fraction (RA-CF, 102 g). The remaining RA-T was then treated with n-butanol (1:1 by volume) three times; the resulting solvent extracts were pooled together and concentrated, thereby yielding the butanol fraction (RA-BU, 107 g). The residual portion of the crude extract was then concentrated, thereby yielding the water fraction (RA-WA, 720 g).

The RA-WA fraction (720 g) was further fractionated using D101 macroporous resin chromatography. The macroporous resin (D101) pre-washed with ethanol and equilibrated with water was packed into a column (10×80 cm). The RA-WA fraction (700 g) was dissolved in water (˜2 L) and loaded on the column. The column was then eluted consecutively with H₂O (˜42 L), 60% EtOH/H₂O (˜35 L) and 96% EtOH/H₂O (30 L). Two fractions, RA-WA1 (eluent from H₂O) and RA-WA2 (combined eluents from 60% EtOH/H₂O and 96% EtOH/H₂O), were obtained with weight of 607 g and 77 g, respectively.

The RA-BU fraction was fractionated using D101 macroporous resin chromatography. The RA-BU (100 g) fraction was dissolved in 500 mL of 20% EtOH/H₂O and loaded on pre-packed column. The column was consecutively eluted with H₂O (˜6 L), 30% EtOH/H₂O (˜25 L), 60% EtOH/H₂O (˜20 L), and 96% EtOH/H₂O (10 L). Four fractions, RA-BU1, RA-BU2, RA-BU3 and RA-BU4, were produced accordingly.

The fraction RA-WA1 was separated into two fractions by ethanol precipitation. RA-WA1 (˜138 g) was dissolved in ˜300 mL of H₂O and 96% ethanol/H₂O (˜600 mL) was added slowly by constantly stirring and the solution was allowed to stand overnight at room temperature to separate into two layers. The supernatant layer and the precipitated residue were separated by filtration. The solution was subjected to second precipitation by adding another 300 mL portion of 96% ethanol/H₂O. The second batch of supernatant layer and the precipitated residue were obtained and two batches of each solution and residue were combined together. This process was carried out for three times and three supernatant layers were combined and dried as RA-WA1-1 and the precipitates were combined and dried as RA-WA1-2 (42 g).

The fraction of RA-WA1-2 was fractionated by SEPHADEX® LH-20 column chromatography. The SEPHADEX® LH-20 column was packed and equilibrated with H₂O. The RA-WA1-2 (11 g) was dissolved in ˜10% MeOH/H₂O (22 mL) and the solution was gently loaded onto the column. The column was initially eluted by 10% MeOH/H₂O with consecutively increasing of MeOH to 20% MeOH/H₂O, 30% MeOH/H₂O, 40% MeOH/H₂O and 50% MeOH/H₂O. 13 fractions were obtained from this chromatography and could be used for further purification.

The fraction RA-BU-4 was fractionated by silica gel (Merck, 0.04-0.063 μm) column chromatography. RA-BU-4 was dissolved in MeOH and mixed with coarse silica gel (0.063-0.20 μm), to yield a dried sample by the solvent evaporation. The dried sample was loaded on column and eluted with 5% MeOH/CHCl₃, with the gradient increased to 10%, 20%, 50%, and 100% MeOH. The eluting fractions were monitored and combined based on TLC analysis; a total of 42 fractions were obtained.

### Analysis of Fractions

Given the high polarity of fractions obtained from RA-WA, HPLC with conventional reverse phase C-18 column is no longer suitable for its analysis. Hydrophilic Interaction Liquid Chromatography (HILIC) is an alternative and straightforward chromatography technique for separation of polar and hydrophilic compounds. The fractions RA-WA, RA-WA1, RA-WA2, RA-WA1-1, RA-WA1-2, and RA-WA1-2-1 to RA-WA1-2-13 were analyzed using Cosmosil HILIC packed column (4.6×150 mm), PDA detector, and gradient of Acetonitrile/H₂O as mobile phase, and the chromatography conditions were optimized for preparative scale purification.

### Compounds Isolation

The sub-fraction RA-BU4-33 was further purified by SEPHADEX® LH-20 column chromatography. Sample was dissolved in MeOH (˜2 mL) and gently loaded on pre-equilibrium column and eluted by MeOH.

The fourth fraction, RA-BU4-33-4, was obtained as pure component, designated as G192-C07, based on TLC analysis. The structure of compound G192-C07 was elucidated by using of NMR spectroscopy, MS spectrometry and comparison with the literature data.

The fractions of RA-WA1-2-08, RA-WA1-2-09, RA-WA1-2-10, RA-WA1-2-11, RA-WA1-2-12 and RA-WA1-2-13 were further separated by preparative Cosmosil HILIC packed column (20×250 mm). The peak collections were corresponded to the UV absorption and the retention time (Rₜ) of the chromatogram. Compounds G192-C16, G192-C17 and G192-C18 from RA-WA1-2-08; G192-C16 and G192-C17 from RA-WA1-2-09; G192-C19, G192-C23, G192-C28 and G192-C29 from RA-WA1-2-10; G192-C22 and G192-C25 from RA-WA1-2-11; G192-009 and G192-C11 from RA-WA1-2-12; G192-C10 and G192-C15 from RA-WA1-2-13, were isolated and identified.

All isolated compounds were analyzed and their structures were elucidated by means of NMR spectroscopy, MS spectrometry and comparison with the reported data.

### Analysis of Physical Properties of Compounds

¹H, ¹³C and 2D NMR spectra was recorded on Varian Mercury 300 and 500 MHz NMR spectrometer. Compounds were dissolved in CD₃OD or DMSO-d₆, or CD₃OD/H₂O (3-10 mg/mL) and all spectra were acquired at room temperature. Chemical shifts were reported as ppm using the solvent peak as a reference (¹H 3.31 ppm and ¹³C 49.05 ppm for CD₃OD, ¹H 2.50 ppm, ¹³C 39.50 ppm for DMSO-d₆) Finnigan MAT LCQ was used for acquiring ESI-MS spectra at Sheath gas 60 psi, auxiliary gas 20 psi, spray voltage 4.5 KV and capillary voltage 30 V. Both positive and negative mode were used for acquiring mass spectra at represented by [M+H]⁺, [M+Na]⁺ or [M−H]⁻ at room temperature. Optical rotation was measured with PERKIN-ELMER 241 polarimeter in MeOH/H₂O at room temperature.

Column chromatography was performed with macroporous resin (D101, Tianjin, China) and SEPHADEX® LH-20 (40-70 μm, Amersham Pharmacia Biotech AB, Uppsala, Sweden). Preparative HPLC was carried out on a Waters 2545 Binary Gradient Module pump system equipped with 2996 Photodiode Array Detector using Cosmosil HILIC packed column (20×250 mm i.d.). The analytical HPLC was performed at Waters system with 2996 Photodiode Array Detector using Cosmosil HILIC packed column (4.6×150 mm i.d., 4.6 μm).

Cellular Cytotoxicity Measurement using LDH Assay

Adult keratinocytes were maintained in Epilife (Cascade Biologics) medium supplemented with human keratinocyte growth supplement (HKGS, Cascade Biologics). On day 0, keratinocytes were seeded at a density of 2.5×10⁵ cells/well in a 48-well plate (Falcon). After an overnight incubation at 5% CO₂ and 37° C., total extract (RA-T), butanol (RA-BU), chloroform (RA-CF), and water (RA-WA) fractions of Rhizoma arisaematis (100 μg/mL) were added to the cells. Epidermal growth factor (EGF, Sigma) at 50 ng/mL and DMSO (0.1%) were used as a positive and a vehicle control, respectively.

After an overnight incubation in 5% CO₂ and 37° C., cytotoxicity detection was performed using a lactate dehydrogenase (LDH) kit according to the manufacturer's instruction (Roche). The LDH level was detected by spectrophotometric microtiter plate reader at 490 nm wavelength.

### Immunoblotting

Human adult fibroblasts in M106 medium (Cascade Biologics) with low serum growth supplement (LSGS, Cascade Biologics) were seeded at a density of 1×10⁷/plate in 60 mm plates (Falcon) and incubated overnight at 5% CO₂ and 37° C. for 24 h. On day 2, the existing media were replaced with 0.2% LSGS medium and each plate was treated with 100 gg/mL of extracts and fractions of Rhizoma arisaematis. Fibroblast growth factor (FGF, Sigma) at 50 ng/mL and DMSO (0.1%) were used as a positive and vehicle control, respectively.

On day 3, cell lysates were obtained by centrifugation at 14000 rpm at 4° C. for 10 min.

Protein concentration of the samples were determined. Equal amounts of proteins (30 μg) and collagen type I (300 ng, Invitrogen) were loaded and run on 10% SDS-PAGE and transferred to nitrocellulose membranes. The membranes were blocked in non-fat milk (5% milk in TBS, 0.05% Tween-20) for 2 h, incubated with Collagen I primary antibody (R and D Systems) overnight at 4° C., and washed three times for 10 min each with TBS (0.5% Tween-20). Membranes were then incubated with rabbit HRP-conjugated secondary antibody (Cell Signaling) and washed as described above. The membranes were then transferred and developed using ECL Western blotting kit (GE Healthcare UK Ltd).

### In-vitro Scratch Wound-Healing Assay

Human neonatal or adult keratinocytes late population doubling time (PD≧15) were trypsinized and seeded onto 6-well tissue culture plates (Falcon) at a density of 3×10⁶ cells/well in HKGS (Cascade Biologics) and EpiLife (Cascade Biologics). After an overnight incubation in a 5% CO₂ and 37° C. chamber, a bisecting scratch was made with a sterile p1000 micropipette tip and the cells were washed with PBS twice, and then replaced with the basal medium without the supplement for the duration of the assay.

Total extract, fractions and compounds of Rhizoma arisaematis were diluted in the basal medium without the supplement and added to the wells. EGF (Sigma) at 50 ng/mL was used as positive control, and DMSO (0.1%) was used as a vehicle control.

For each scratch, two consecutive fields were selected using the following criteria: relatively little cell debris within the scratch; even scratch, with straight edges; both edges visible under a single field using the 10× objective; fields not too close to either end of the scratch. The pictures were taken from day 1 for five consecutive days. The medium and the extracts were replenished daily for the duration of the assay.

### Proliferation Assay

Human neonatal keratinocytes of early population doubling time (PD≦8) were seeded onto 96-well tissue culture plates (Falcon) at a density of 1000 cells/well in HKGS (Cascade Biologics) and Epilife (Cascade Biologics) basal medium. After an overnight incubation in a 37° C. and 5% CO₂ chamber, the cells were washed with PBS twice then replaced with the basal medium without the supplement for the duration of the assay.

Total extract, fractions and compounds of Rhizoma arisaematis were diluted in the basal medium without the supplement and added to the wells in triplicates. EGF (Sigma) at 50 ng/mL was used as a positive control and DMSO (0.1%) was used as a solvent control. The treated cells were then returned to a 37° C. and 5% CO₂ chamber for 48 hours.

MTT (3[4,5-dimethylthiazol-2-yl]-2,5-diphenyltetrazolium bromide) metabolic assay (USB) was used to monitor the proliferative capacity of the cells according to the manufacturer's protocol. MIT level was detected by spectrophotometric microtiter plate reader at 570 nm wavelength on day 3, day 4, and day 5 after treatment.

In vivo Incision Wound Healing Assay

All experiments were carried out on 20-25 g male “imprinting control region” (ICR) mice obtained from The Hong Kong University of Science and Technology Animal Care Facility. The study was approved by the HKUST Animal Ethics Committee and conducted in accordance with the Code of Practice for Care and Use of Animals for Experimental Purposes.

Briefly, one month old ICR mice were anesthetized by i.p. injection with chloral hydrate. Two full-thickness wounds were generated on the shaved backs of each mouse using a 6-mm diameter sterile puncher as described in Gal et al., 2008 Vet. Med., 52: 652-659. After wounding, each mouse received Rhizoma arisaematis extracts on one wound, and the other wound was left untreated. Each mouse was then housed individually. The mice received treatment twice a day. Each wound was photographed using a digital camera every 2 days. The wounded areas in the images were measured and compared to the vehicle-treated control.

### EXAMPLES

Following are examples that illustrate embodiments for practicing the invention. These examples should not be construed as limiting. All solvent mixture proportions are by volume unless otherwise noted.

### Example 1

### Water Fraction of Rhizoma Arisaematis Closes in Vitro Scratch Wounds in Adult Human Keratinocytes

The proliferation and migration of keratinocytes is required for the closure of wound bed. To identity therapeutic agents with wound-healing activity, it is important to evaluate whether the agent facilitates the migration and/or proliferation of keratinocytes.

To examine the efficacy of the herb Rhizoma arisaematis (RA) in in vitro closure of scratch wounds, human keratinocytes were incubated with the crude extract (RA-T) or different fractions of RA (100 μg/mL).

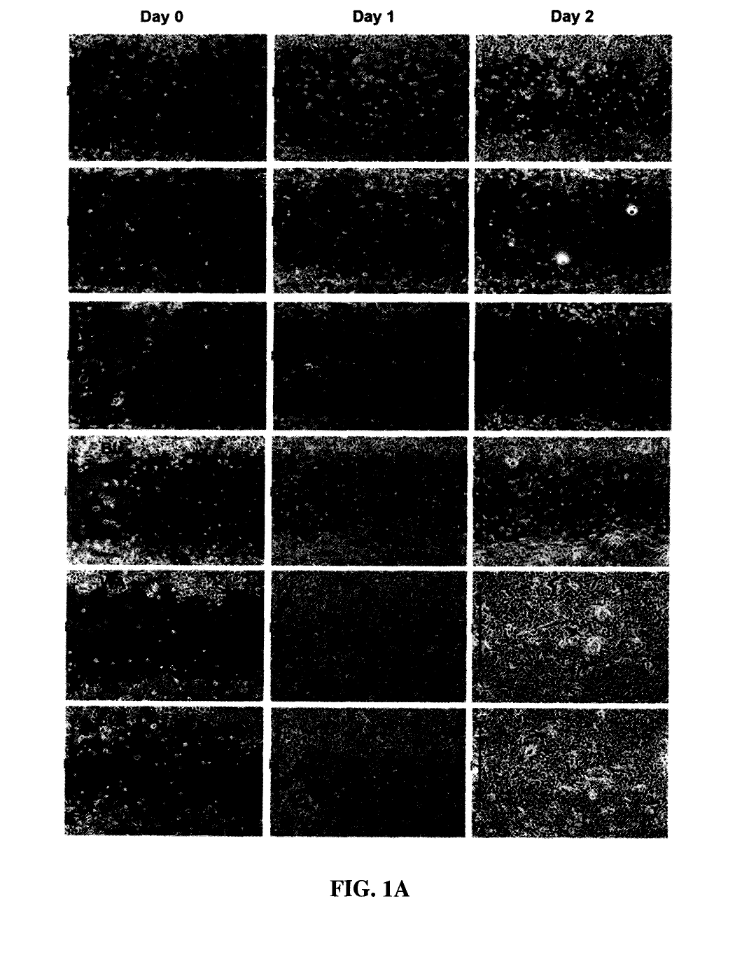

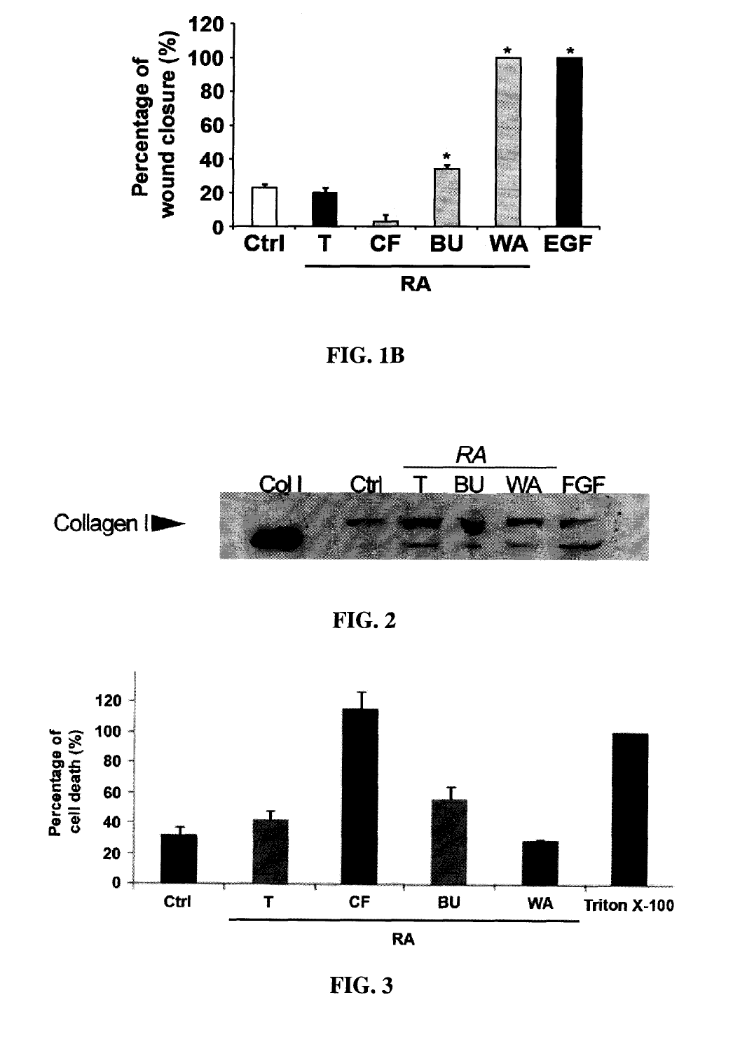

As shown in FIG. 1, the water fraction (RA-WA) and the positive control, epidermal growth factor (EGF, 50 ng/mL), exhibited the highest wound-healing effects and closed the wounds by day 2. The butanol fraction (RA-BU) also exhibited wound-healing activity. Scratch wounds treated with the crude extract (RA-T) and the chloroform fraction (RA-CF) remained open throughout the assay.

### Example 2

### The Water Fraction and the Crude Extract of Rhizoma Arisaematis Induce Collagen Type I Production in Human Adult Fibroblasts

The formation of collagen matrices by fibroblasts is a critical step in the wound-healing process. After the initial phase of pro-inflammatory responses induced by immune cells surrounding the wound, fibroblasts begin to proliferate and migrate around and over the wound bed. The proliferating fibroblasts produce collagen that forms matrices covering the wound bed. The matrices prevent further injuries from the wound area and act as a scaffolding structure for the tissue repair process including angiogenesis and construction of connective tissue. Currently, therapeutics that can increase collagen production in human fibroblasts are limited to platelet-derived growth factor (PDGF) and certain types of acids.

Human adult fibroblasts were incubated with the fractions of RA (100 μg/mL). Fibroblast growth factor (FGF, 50 ng/mL) was used as a positive control. Purified collagen type I protein (Col I, 300 ng, Invitrogen) was used as an external positive control and DMSO at 0.1% (Ctrl) was used as a vehicle control. Cell lysates were obtained after 24 hour treatment and the protein concentration of the samples was determined. Equal amounts of proteins (30 μg) and collagen type I were loaded and run on 10% SDS-PAGE and transferred to nitrocellulose membranes. The membranes were incubated with rabbit HRP-conjugated secondary antibody (Cell Signaling). The membranes were then transferred and developed for analysis.

As shown in FIG. 2, the total extract and the WA fractions of RA increased the expression of collagen type I in human adult fibroblasts. The positive control, fibroblast growth factor (FGF), increased the collagen expression marginally. Collagen type I is the most abundant collagen type present in humans.

### Example 3

### Water Fraction and Crude Extract of Rhizoma Arisaematis do not Cause Cell Death in Human Neonatal Keratinocytes

The lactate dehydrogenase (LDH) assay is a sensitive and reliable screening method for evaluating cell death caused by exogenous treatment of pharmaceutical compounds. LDH is a cytoplasmic enzyme that is present in all cells and is released into supernatants upon breakdown of the plasma membrane.

The effects of the RA extract and fractions on neonatal keratinocytes were examined. Human neonatal keratinocytes were treated with fractions of RA (100 μg/mL). After overnight incubation, lactate dehydrogenase (LDH) released into the medium was measured. Cell death was calculated as a percentage compared to the vehicle control (0.1% DMSO, Ctrl). Triton-X 100 at 0.1% was used as a positive control. Triton-X 100 is a nonionic surfactant that ruptures cell membranes and causes cell death. Assay was conducted in duplicate.

As shown in FIG. 3, while the RA-CF and RA-BU fractions caused severe and mild cell death, respectively, the crude extract (RA-T) and water fraction (RA-WA) did not result in cell death when compared to the vehicle control (Ctrl, 0.1% DMSO).

### Example 4

The water fraction of Rhizoma arisaematis Increases the Proliferation of Human Neonatal Keratinocytes

Proliferation of keratinocyte is another important component of wound healing process, where closure of the wound bed is facilitated by the migration and proliferation of keratinocytes.

The effect of the RA extract and fractions on proliferating human keratinocytes was examined. The proliferative capacity of keratinocytes was measured using the MIT (3-[4,5-dimethylthiazol-2-yl]-2,5-diphenyltetrazolium bromide) metabolic assay according to the manufacturer's protocol. MIT is reduced in living cells by mitochondrial dehydrogenase to a blue formazan product, which can be eluted and quantified in a spectrophotometer. The assays were conducted in duplicate and repeated twice. Values are expressed as mean±SEM.*denotes p<0.05 when compared to control (Ctrl), Student t-test.

Specifically, human neonatal keratinocytes were seeded onto 96-well tissue culture plates at a density of 1000 cells/well in growth supplement with basal medium. After overnight incubation, RA extracts (100 μg/mL) diluted in the basal media without the supplement were added to the wells in triplicate. Epidermal growth factor (EGF) (50 ng/mL) was used as the positive control and 0.1% DMSO was used as the vehicle control (Ctrl).

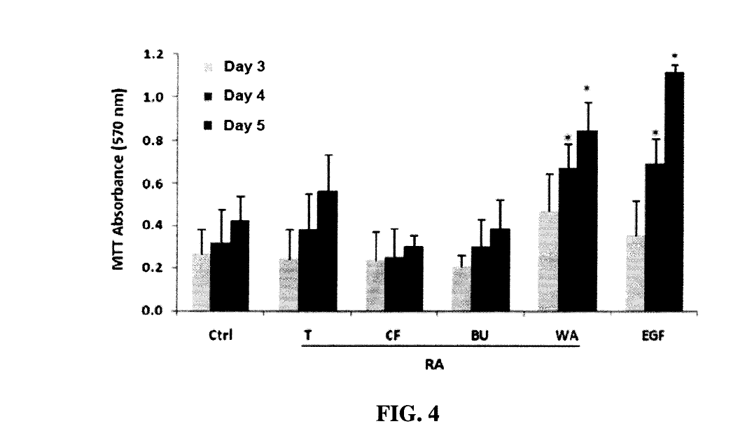

As shown in FIG. 4, the RA-WA fraction (100 μg/mL) increased the proliferation of keratinocytes significantly on day 3, when compared to the vehicle control (Ctrl, 0.1% DMSO). EGF (50 ng/mL) was used as a positive control. EGF is a known proliferative agent of keratinocytes.

### Example 5

The water Sub-Fractions of Rhizoma arisaematis Close in vitro Scratch Wounds in Adult Human Keratinocytes.

To examine the efficacy of the WA sub-fractions of RA in closing in vitro scratch wounds, RA-WA1 and RA-WA2 (1 μg/mL and 10 μg/mL) were tested using human keratinocytes. The scratch assay was performed using the procedures as described in Example 1. RA-WA1 and RA-WA2 at 1 μg/mL and 10 μg/mL were evaluated for their ability to close in vitro wound gap.

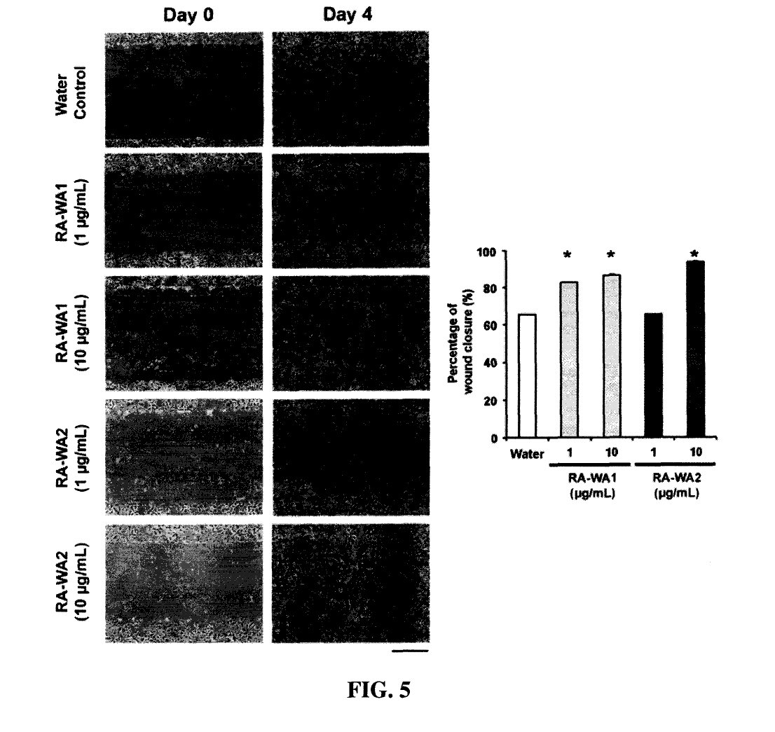

As shown in FIG. 5, RA-WA1 at both 1 and 10 μg/mL, and RA-WA2 at 10 μg/mL closed the wounds by day 4. Scratch wounds treated with RA-WA2 at 1 μg/mL and the vehicle control (water) remained open throughout the assay. Left panel: representative pictures of cell gaps taken after a 4-day treatment; Right panel: wound closure was quantified and presented as percentage of wound closure following various treatments. The assays were repeated three times. Values are expressed as mean±SEM.*denotes p<0.05 when compared to control (Water), Student t-test.

### Example 6

### The Water Sub-Fraction of Rhizoma Arisaematis Promotes Wound Closure in Mouse Incision Wound Model

One month old “imprinting control region” (TCR) mice were anesthetized by i.p. injection with chloral hydrate. Two full-thickness wounds were generated on the shaved backs of each mouse using a 6-mm diameter sterile puncher. After wounding, mice were separated into four groups (with three mice in each group) as follows: first group: each mouse was treated with the WA fraction at 4 mg/kg on one wound by topical administration, and the other wound was left untreated; second group: each mouse was treated with the WA fraction at 40 mg/kg on one wound by topical administration, and the other wound was left untreated; third group: each mouse was treated with the sub-fraction RA-WA1 at 4 mg/kg on one wound by topical administration, and the other wound was left untreated; fourth group: each mouse was treated with the sub-fraction RA-WA1 at 40 mg/kg on one wound by topical administration, and the other wound was left untreated; fifth group: each mouse was treated with water (10 μl) on one wound by topical administration, and the other wound was left untreated. Every mouse was then housed individually, and treated with the corresponding fraction or sub-fraction of the RA extract twice a day. Each wound was photographed using a digital camera every 2 days for 8 days. Progress in wound closure was traced and the wounded areas were quantified.

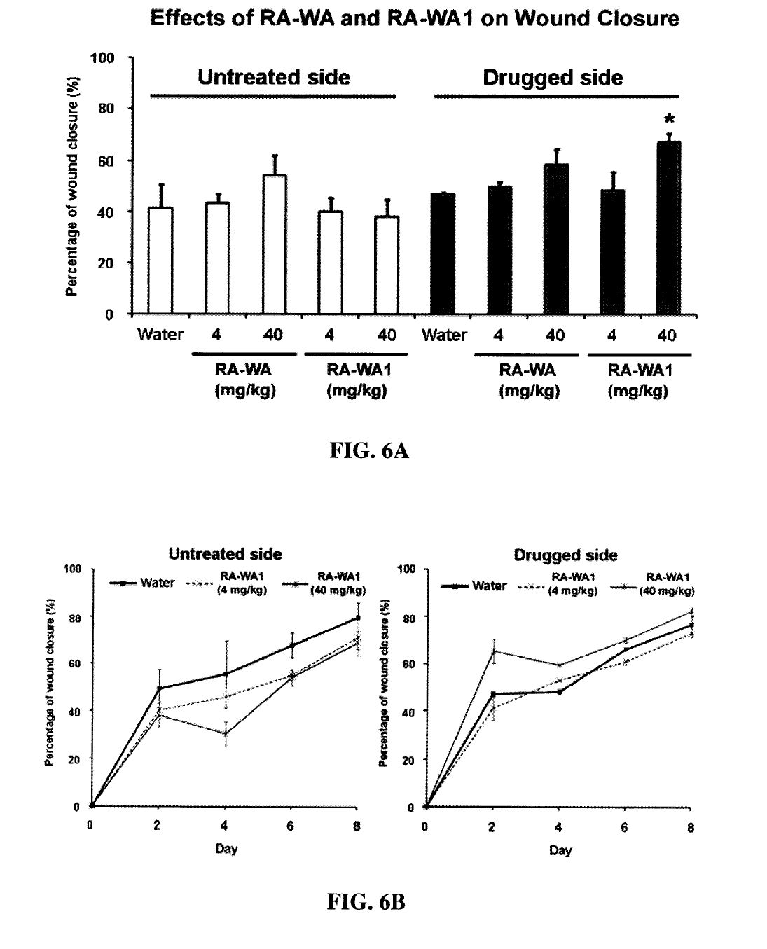

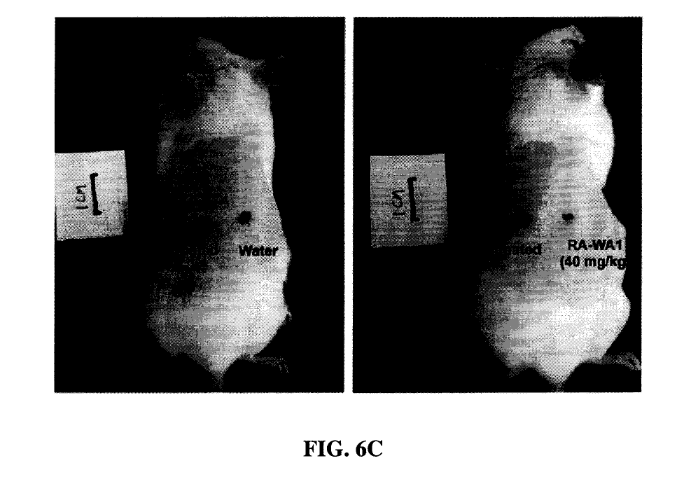

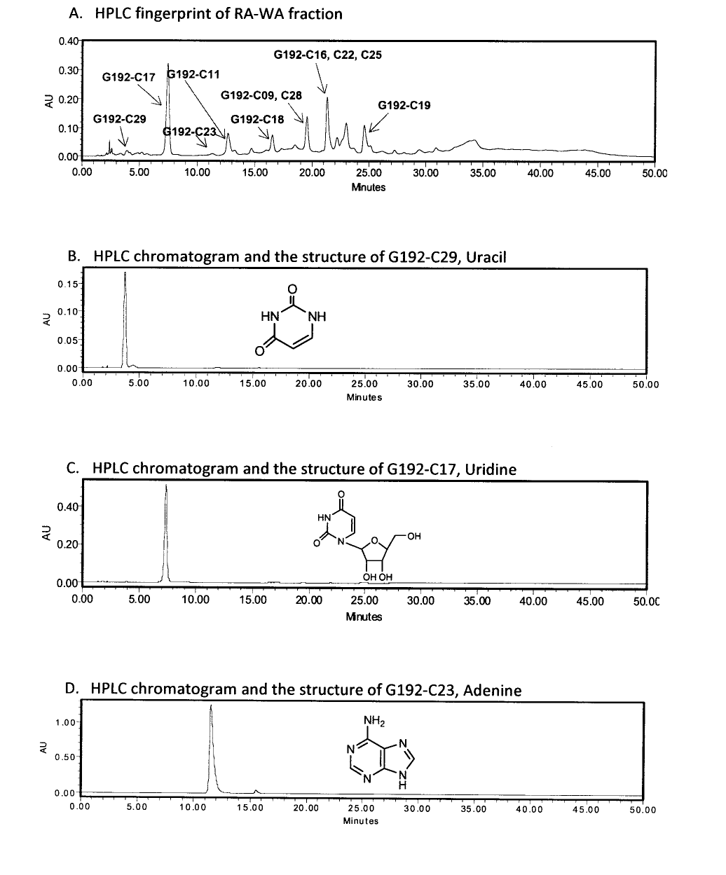

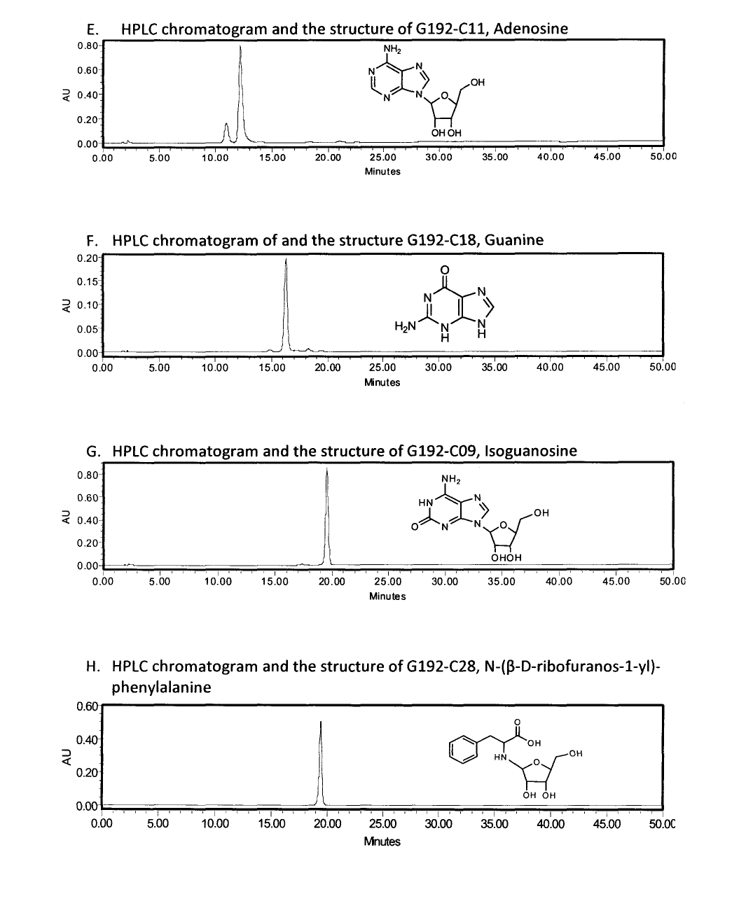

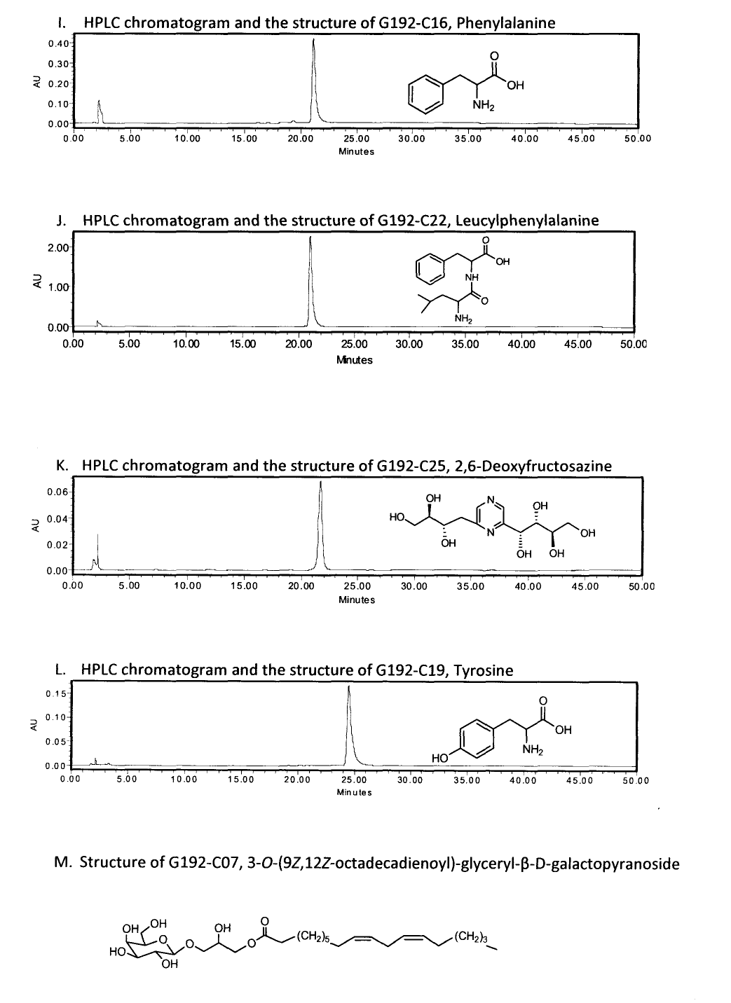

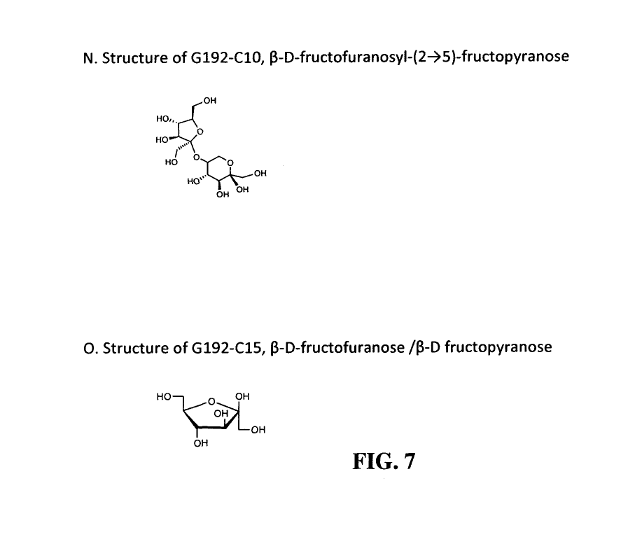

FIG. 6 shows that the WA subfraction, RA-WA1, promoted wound closure in mouse incision wound model. FIG. 6A shows that the topical administration of the RA-WA fraction and RA-WA1 promoted wound closure on day 2. RA-WA1 significantly promoted wound closure in mouse model. Values are expressed as mean±SEM.*denotes p<0.05 when compared to control (Water), Student t-test. FIG. 6B shows that the topical administration of RA-WA1 at 4 and 40 mg/kg promoted wound closure. The wound closure in mice treated with RA-WA1 at 40 mg/kg was faster than in those treated with a lower concentration of RA-WA1 (4 mg/kg) and water during the whole process of 8-day study. Values are expressed as mean±SEM.

FIG. 6C shows the pictures of mice treated with RA-WA1 40 mg/kg and water by topical administration on day 8.

### Example 7

### Hplc Profiling Of RA-WA Fraction and the Isolated Compounds

A HPLC chromatographic method was developed to profile the WA fraction, subfractions (RA-WA1 and RA-WA1-2) and the isolated compounds. A HILIC, Hydrophilic Interaction chromatography column (4.6×150 mm) was used as stationary phase, while gradient of acetonitrile (ACN) and water were used as mobile phase, started with 95% ACN/5% H₂O and increased to 70% ACN/30% H₂O in 35 min, continually up to 20% ACN/80% H₂O in 45 min, and to 95% ACN/5% H₂O in 50 min (See table 1). The photodiode array detector was used as detector.

All RA-WA fraction and isolated compounds were analyzed at the same HPLC conditions, and UV absorbance was detected at 254 nm. The HPLC profiles of the fraction RA-WA and isolated compounds as well as their structures were shown in FIG. 7. Compared the retention time of RA-WA with those of isolated compounds, total 11 peaks were identified.

TABLE 1

| Time (min) | ACN (%) | H₂O (%) |
|---|---|---|
| 0 | 95 | 5 |
| 35 | 70 | 30 |
| 45 | 20 | 80 |
| 50 | 95 | 5 |

### Example 8

Isolation of Compounds from Rhizoma arisaematis

Various compounds were isolated from the Rhizoma arisaematis extracts and fractions.

Compound G192-C07 was obtained as light yellow oil. The molecular formula of G192-C07 is C₂₇H₄₈O₉ based on a molecular ion at m/z [M+Na]⁺ 539.2 in positive ESI-MS. The structure of G192-C07 was identified by NMR and MS as 3-O-(9Z,12Z-octadecadienoyl)-glyceryl-β-D-galactopyranoside.

¹H-NMR (300 and 500 MHz, CD₃OD, ppm)δ: 4.22 (d, 8.2, Gal-1), 3.53 (m, Gal-2), 3.51 (m, Gal-3), 3.83 (d, 2.7, Gal-4), 3.49 (m, Gal-5), 3,74 (2H, m, Gal-6), 3.92, 3.66(2H, H-1′), 3.99 (1H, t, 4.8, H-2′), 4.14 (2H, dd, 5.4, 1.2, H-3′), 2.33 (2H, t, 7.5, H-2), 1.60 (2H, m, H-3), 1.3 (8H, m, H-4,5,6,7), 2.05 (2H, m, 5.35, 5.33(4H, m, H-9,10, H-12,13), 2.78 (2H, m, H-11), 1.3 (4H, m, H-14,15), 1.33 (2H, m, H-16), 1.26 (2H, m, H-17), 0.9 (3H, m, H-18).

¹³C-NMR (75 MHz, CD₃OD, ppm)δ: 105.2 (Gal-1), 72.5 (Gal-2), 76.7 (Gal-3), 70.2 (Gal-4), 74.7 (Gal-5), 62.4 (Gal-6), 71.8 (C-1′), 69.5 (C-2′), 66.5 (C-3′), 175.4 (C-1), 130.9, 129.1, 130.8, 129.0 (C-9,10, C-12,13), 34.9 (C-2), 25.9 (C-3), 30.7-30.2 (C-4,5,6,7 and C-14,15), 28.1 (C-8), 26.5 (C-11), 32.6 (C-16), 23.6 (C-17), 14.5 (C-18).

Compound G192-C09 was obtained as white powder and UV absorption was at λₘₐₓ (CH₃CN): 253.2 nm. The molecular formula of G192-C09 is C₁₀H₁₃N₅O₅ based on a molecular ion at m/z [M+H]+284.00, [2M+H]⁺ 566.92 and [2M+Na]⁺ 589.11 in positive ESI-MS. The structure of G192-C09 was identified by NMR and MS as 2-hydroxyadenosine (Isoguanosine).

¹H-NMR (300 and 500 MHz, CD₃OD, ppm) δ: 7.97(1H, s, H-8), 5.85 (1H, d, J 6.0 Hz, H-1′), 4.65 (1H, H-2′), 4.34 (1H, m, H-3′), 4.17 (1H, H-4′), 3.86, 3.77 (2H, dd, H-5′). ³³C-NMR (75 MHz, CD₃OD, ppm) δ: 156.1 (C-2), 147.6 (C-6), 142.9 (C-8), 120.0 (C-4), 116.2 (C-5), 90.7 (C-1′), 75.8 (C-2′) 71.7 (C-3′), 87.3 (C-4′), 62.6 (C-5′).

Compound G192-C10 was obtained as light yellow gel. The molecular formula of G192-C10 is C₁₂H₂₂O₁₁ based on a molecular ion at m/z [M+H]⁺ 343.55 in positive ESI-MS. The structure of G192-C10 was identified by NMR and MS as β-D-fructofuranosyl-(2→5)-fructopyranose.

¹H-NMR (300 MHz, CD₃OD/D₂O, ppm) δ: 4.05, 4.04, 4.00, 3.83, 3.78, 3.73, 3.70, 3.68, 3.64, 3.62, 3.49, 3.48, 3.47, 3.45. ¹³C-NMR (75 MHz, CD₃OD/D₂O, ppm) β: 103.2, 99.2, 83.3, 77.5, 76.8, 71.9, 71.3, 69.4, 65.9, 64.8, 64.5, 64.3.

Compound G192-C11 was obtained as white powder and UV absorption was at λₘₐₓ (CH₃CN): 259.1 and 207.1 nm. The molecular formula of G192-C11 is C₁₀H₁₃N₅O₄ based on a molecular ion at m/z [M+H]⁺ 268.32 in positive ESI-MS. The structure of G192-C11 was identified by NMR and MS as adenosine.

¹H-NMR (300 and 500 MHz, DMSO-d₆, ppm) δ: 8.10 (1H, s, H-2), 8.08 (1H, s, H-8), 5.86 (1H, d, J 6.0Hz, H-1′), 4.60 (1H, H-2′), 4.14 (1H, m, H-3′), 3.96 (1H, H-4′), 3.68, 3.65 (2H, dd, ¹³C-NMR (75 MHz, DMSO-d₆, ppm) δ: 156.2 (C-6), 151.9 (C-2), 148.7 (C-4), 139.2 (C-8), 118.1 (C-5), 88.1 (C-1′), 72.9 (C-2′), 72.1 (C-3′), 85.6 (C-4′), 61.5 (C-5′).

Compound G192-C15 was obtained as achromatic gel. The molecular formula of G192-C15 is C₆H₁₂O₆ based on a molecular ion at m/z [M+Na]⁺ 203.61 in positive ESI-MS. The structure of G192-C15 was identified by NMR and MS as fructose which consisted of both β-D-fructofuranose and β-D-fructopyranose.

¹H-NMR (300 MHz, CD₃OD/D₂O, ppm) δ: 4.06, 3.99, 3.85, 3.79, 3.75, 3.64, 3.49. ¹³C-NMR (75 MHz, CD₃OD/D₂O, ppm) δ: 102.8, 71.1, 71.1, 69.2, 65.7, 65.4.

Compound G192-C16 was obtained as white powder and UV absorption was at λₘₐₓ (CH₃CN): 209 nm. The molecular formula of G192-C16 is C₉H₁₁NO₂ based on a molecular ion at m/z [M+H]⁺ 166.48 in positive ESI-MS and m/z [M−H]⁻ 164.43 in negative mode. The structure of G192-C16 was identified by NMR and MS as 2-amino-3-phenylpropanoic acid (Phenylalanine).

¹H-NMR (300 MHz, CD₃OD/D₂O, ppm) δ: 8.10 (1H, s, H-2), 7.14-7.26 (5H, m, aromatic protons), 3.77 (1H, dd, H-2′), 3.14, 2.92 (2H, dd, H-1′). ¹³C-NMR (75 MHz, CD₃OD/D₂O, ppm) δ: 174.5 (C-3′), 136.3 (C-1′), 130.4, 130.1 (C-2,6 and C-3,5), 128.6 (C-4), 57.5 (C-2′), 37.6 (C-1′).

Compound G192-C17 was obtained as pale yellow paste and UV absorption was at λₘₐₓ (CH₃CN): 257.9 and 207.1 nm. The molecular formula of G192-C17 is C₉H₁₂N₂O₆ based on a molecular ion at m/z [M−H]⁻ 243.43 in negative mode ESI-MS. The structure of G192-C17 was identified by NMR and MS as uracil-β-D-ribofuranoside (Uridine).

¹H-NMR (300 MHz, CD₃OD, ppm) δ: 8.01 (1H, d, J=8.4Hz, H-4), 5.70 (1H, d, J=8.4Hz, H-5), 5.89 (1H, d, J=4.2Hz, H-1′), 4.20 (1H, H-2′), 4.15 (1H, m, H-3′), 4.01 (1H, H-4′), 3.85, 3.74 (2H, dd, H-5′). ¹³C-NMR (75 MHz, CD₃OD, ppm) δ: 166.2 (C-6), 152.4 (C-2), 142.5 (C-4) , 102.7 (C-5), 90.6 (C-1′), 75.4 (C-2′), 71.1 (C-3′), 86.3 (C-4′), 62.1 (C-5′).

Compound G192-C18 was obtained as white powder and UV absorption was at λₘₐₓ (CH₃CN): 253.2 and 273.3 nm. The molecular formula of G192-C18 is C₅H₅N₅O based on a molecular ion at m/z [M+H]⁺ 152.1 and [M+K]⁺ 189.9 positive ESI-MS. The structure of G192-C18 was identified by NMR and MS as 2-amino-6-hydroxypurine (Guanine).

¹H-NMR (300 MHz, CD₃OD/H₂O, ppm) δ: 8.46(1H, s, H-8). ¹³C-NMR (75 MHz, CD₃OD/H₂O, ppm) δ: 162.4, 153.7, 145.5, 152.7, 116.3.

Compound G192-C19 was obtained as white powder and UV absorption was at λₘₐₓ (CH₃CN): 224.8 and 276.9 nm. The molecular formula of G192-C18 is C₉H₁₂NO₃ based on a molecular ion at m/z [M+H]⁺ 182.33 in positive mode of ESI-MS. The structure of G192-C18 was identified by NMR and MS as 2-amino-3-(4-hydroxyphenyl)-propanoic acid (Tyrosine).

¹H-NMR (300 MHz, DMSO-d₆, ppm) δ: 7.15 (2H, d, H-2,6), 6.79 (2H, d, H-3,5), 3.75 (1H, dd, H-2′), 3.19, 2.95 (2H, dd, H-1′). ¹³C-NMR (75 MHz, DMSO-d₆, ppm) δ: 181.4 (C-3′), 156.0 (C-4), 127.5 (C-1), 130.2 (C-2,6), 115.1 (C-3,5), 55.9 (C-2′), 42.9 (C-1′).

Compound G192-C22 was obtained as white powder and UV absorption was at λₘₐₓ (CH₃CN): 207 nm. The molecular formula of G192-C22 is C₁₅H₂₂N₂O₃ based on a molecular ion at m/z [M+H]⁺ 279.17 and m/z [M+Na]⁺ 301.14 in positive mode of ESI-MS. The structure of G192-C22 was identified by NMR and MS as leucylphenylalanine.

¹H-NMR (300 and 500 MHz, CD₃OD/D₂O, ppm) δ: 7.31-7.40 (5H, m, H-2 to H-5), 3.71 (1H, dd, H-2′), 3.29, 3.10 (2H, dd, H-1′), 3.96 (1H, d, H-2″), 1.75, 1.66 (2H, m, H-3″), 1.74 (1H, H-4″), 0.98, 0.97 (6H, t, 2×CH₃). ¹³C-NMR (75 MHz, CD₃OD/D₂O, ppm) δ: 136.3 (C-1), 128.6(C-4), 130.0 (C-2,6), 130.3 (C-3,5), 54.2(C-2% 37.5 (C-1′), 174.3 (C-1′), 175.8 (C-1″), 57.0 (C-2″), 40.9 (C-3″), 25.2 (C-4″), 21.9, 23.0 (2×CH₃).

Compound G192-C23 was obtained as white powder and UV absorption was at λₘₐₓ (CH₃CN): 259.1 and 207.1 nm. The molecular formula of G192-C23 is C₅H₅N₅ based on a molecular ion at m/z [M+H]⁺136.50 in positive ESI-MS. The structure of G192-C23 was identified by NMR and MS as adenine.

¹H-NMR (300 MHz, CD₃OD, ppm) δ: 8.20, 8.22 (2H, s, H-2, 8), ¹³C-NMR (75 MHz, CD₃OD, ppm) δ: 155.9 (C-6), 153.0 (C-2), 151.8 (C-4), 142.1 (C-8), 117.8 (C-5).

Compound G192-C25 was obtained as yellow paste and UV absorption was at λₘₐₓ (CH₃CN): 274.5 and 206 nm. The molecular formula of G192-C25 is C₁₂H₂₀N₂O₇ based on a molecular ion at m/z [M+H]⁺ 305.1377 and [M+Na]⁺ 327.1130 in positive mode HR-ESI-MS. The structure of G192-C25 was identified by NMR and MS as 2-(1,2,3,4-tetrahydroxybutyl)-6-(2,3,4-trihydroxybutyl)-pyrazine, 2,6-deoxyfructosazine.

¹H-NMR (300 MHz, CD₃OD, ppm) δ: 8.71 (1H, s, H-5), 8.53 (1H, s, H-3), 5.16 (1H, s, H-1′), 3.87 (1H, m, H-2′), 3.84 (1H, m, H-3′), 3.82, 3.66 (2H, m, H-4′), 3.22, 2.98 (2H, m, H-1″), 4.03 (1H, m, H-2″), 3.70 (1H, m, H-3″), 3.86, 3.67 (2H, m, H-4″). ¹³C-NMR (75 MHz, CD₃OD, ppm) δ: 157.0 (C-2), 156.2 (C-6), 145.1 (C-5), 147.0 (C-3), 74.0 (C-1′), 77.4 (C-2′), 74.3 (C-3′), 65.4 (C-4′), 40.5 (C-1″), 74.2(C-2″), 76.3(C-3″), 65.9 (C-4″).

Compound G192-C28 was obtained as white powder, [α]_{D}²⁰=−9.7 (C 2.36, MeOH/H₂O 1:1) and UV absorption was at λₘₐₓ (CH₃CN): 253.2 nm. The molecular formula of G192-C28 is C₁₄H₁₉NO₆ based on a molecular ion at m/z [M+Na]⁺ 320.0473 and [M+K]⁺ 336.2242 in positive mode of HR-ESI-MS. The structure of G192-C28 was identified by 1D and different 2D NMR spectroscopy like COSY, HSQC, HMBC and MS as N-(β-D-ribofuranos-1-yl)-phenylalanine, which is a novel compound.

¹H-NMR (300 MHz, CD₃OD/D₂O, ppm) δ 7.29-7.39 (5H, m, H-2 to H-5), 3.29, 3.10 (2H, dd, H-1′), 3.84 (1H, d, H-2″), 5.85 (1H, d, J=6 Hz, H-1′), 4.67 (1H, H-2′), 4.34 (1H, m, H-3′), 4.17 (1H, H-4′), 3.79, 3.70 (2H, dd, H-5′). ¹³C-NMR (75 MHz, CD₃OD, ppm) δ: 136.3(C-1), 130.3 (C-3,5), 130.0 (C-2,6), 128.6 (C-4), 57.1(C-2′) 37.5 (C-1′), 174.4 (C-3′), 89.2 (C-1″), 74.7 (C-2″), 71.7 (C-3″), 86.6 (C-4″), 62.6 (C-5″).

Compound G192-C29 was obtained as pale yellow paste and UV absorption was at λₘₐₓ (CH₃CN): 256.7 and 206 nm. The molecular formula of G192-C29 is C₄H₄N₂O₂ based on a molecular ion at m/z [M+H]⁺ 113.21 in negative mode ESI-MS. The structure of G192-C29 was identified by NMR and MS as uracil.

¹H-NMR (300 MHz, CD₃OD, ppm) δ: 7.41 (1H, d, J=7.8 Hz, H-4), 5.61 (1H, d, J=7.8 Hz, H-5). ¹³C-NMR (75 MHz, CD₃OD, ppm) δ: 167.4 (C-6), 152.9 (C-2), 144.6 (C-4), 101.8 (C-5).

### Example 9

### 3-O-(9,12-Octadecadienoyl)-Glyceryl-Beta-D-Galactopyranoside Closes in Vitro Scratch Wounds in Adult Human Keratinocytes

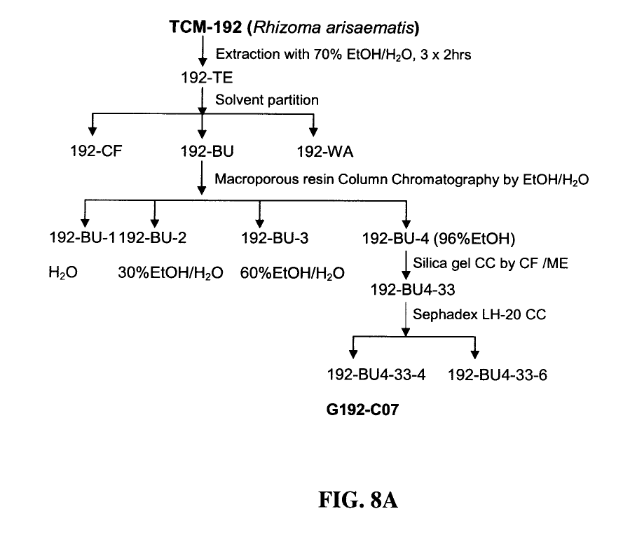

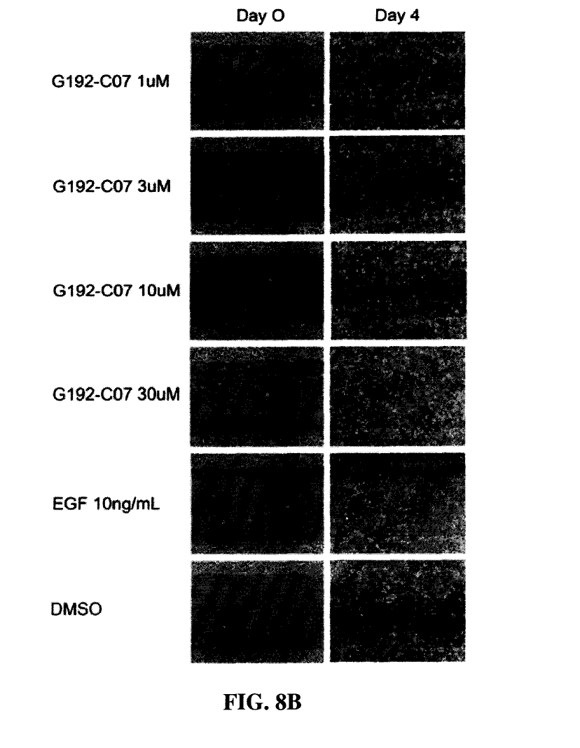

To examine the wound-healing effects, 3-O-(9,12-octadecadienoyl)-glyceryl-β-D-galactopyranoside (G192-C07) isolated from the BU fraction of RA (FIG. 8A) was incubated with human keratinocytes. The scratch assay was performed as described in Examples 1 and 5. G192-C07 at 1, 3, 10, and 30 μg/mL were evaluated for their ability to close in vitro wound gap. Epidermal growth factor (EGF, 10 ng/mL) and 0.1% DMSO were used as a positive and a vehicle control, respectively. Photographs of the wounds were taken immediately after the scratch and on day 4.

As shown in FIG. 8B, G192-C07 at 1, 3, and 10 μM partly closed the wound gap, while G192-C07 at 30 μM and the positive control, epidermal growth factor (EGF, 10 ng/mL), fully closed the wounds by day 4. Scratch wounds treated with the vehicle control (DMSO) remained open throughout the assay.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method for promoting healing of a wound in a subject, comprising administering to the subject, a therapeutically effective amount of a composition comprising a water-soluble Rhizoma arisaematis extract, and/or one or more isolated compounds selected from the group consisting of 3-O-(9,12-octadecadienoyl)-glyceryl-β-D-galactopyranoside, N-(β-D-ribofuranos-1-yl)-phenylalanine, adenosine, and any salts thereof.

2. The method according to claim 1, wherein the composition comprises a water soluble extract of the root of Rhizoma arisaematis.

3. The method according to claim 1, wherein the composition comprises one or more isolated compounds selected from the group consisting of 3-O-(9,12-octadecadienoyl)-glyceryl-β-D-galactopyranoside, N-(β-D-ribofuranos-1-yl)-phenylalanine, adenosine, and any salts thereof.

4. The method according to claim 3, wherein the composition comprises an isolated compound that is 3-O-(9,12-octadecadienoyl)-glyceryl-β-D-galactopyranoside or a salt thereof, and an isolated compound that is adenosine or a salt thereof.

5. The method according to claim 1, wherein the composition is topically administered to a wound area.

6. The method according to claim 1, wherein the subject has a skin wound, excision, laceration, burn, abrasion, penetrating wound, surgical wound, crushing injury, or ulcer.

7. The method according to claim 6, wherein the subject has a diabetic ulcer.

8. The method according to claim 5, wherein the composition is formulated into a wound dressing, cream, ointment, foam, lotion, plaster, gel, emulsion, hydrogel, or skin patch.

9. A method for promoting healing of a wound in a subject, comprising administering to the subject a therapeutically effective amount of a composition comprising an Rhizoma arisaematis extract soluble in a solvent that comprises at least 70% (v/v) alcohol selected from ethanol, methanol, n-propanol, isopropanol, n-butanol, isobutanol, or a mixture thereof.

10. The method according to claim 9, wherein the subject has an ulcer and the composition is topically administered to the ulcer.

11. The method according to claim 10, wherein the subject has a diabetic ulcer.

12. The method according to claim 10, wherein the composition is formulated into a wound dressing, cream, ointment, foam, lotion, plaster, gel, emulsion, hydrogel, or skin patch.

13. A method for promoting collagen production by fibroblasts, wherein the method comprises administering to the fibroblasts an effective amount of a composition comprising a water-soluble Rhizoma arisaematis extract.

14. The method according to claim 13, wherein the fibroblasts are in a subject.

15. The method according to claim 14, wherein the subject has a wound and the composition is topically administered to the wound area.

16. A method for promoting the migration and/or proliferation of keratinocytes, wherein the method comprises administering to the keratinocytes an effective amount of a composition comprising a water-soluble extract of Rhizoma arisaematis and/or an isolated compound that is 3-O-(9,12-octadecadienoyl)-glyceryl-β-D-galactopyranoside or a salt thereof.

17. The method according to claim 16, wherein the keratinocytes are in a subject.

18. The method according to claim 17, wherein the subject has a wound and the composition is topically administered to the wound area.

19. The method according to claim 16, wherein the composition comprises an isolated compound that is 3-O-(9,12-octadecadienoyl)-glyceryl-β-D-galactopyranoside or a salt thereof.

20. An isolated compound that is N-(β-D-ribofuranos-1-yl)-phenylalanine or a salt thereof.
